(19) United States
(12) Patent Application Publication
(10) Pub. No.: US 2022/0125745 A1
(43) Pub. Date: Apr. 28, 2022

(54) PHARMACEUTICAL COMPOSITION FOR CO-ADMINISTRATION OF ACIDOSIS-INDUCING DRUG

(71) Applicant: HAIM BIO CO., LTD., Seoul (KR)

(72) Inventors: Yong Bae KIM, Seoul (KR); Sun Young PARK, Seoul (KR); Kyung Seop YUN, Seoul (KR); Min Hee JANG, Gangwon do (KR); Se Young JEONG, Seoul (KR); Eun Jeong KIM, Seoul (KR); Jin Sam LEE, Gyeonggi do (KR); Hong Yeoul KIM, Seoul (KR)

(73) Assignee: HAIM BIO CO., LTD., Seoul (KR)

(21) Appl. No.: 17/429,791

(22) PCT Filed: Nov. 29, 2019
(86) PCT No.: PCT/KR2019/016729

§ 371 Date: Aug. 10, 2021

(30) Foreign Application Priority Data
Nov. 30, 2018 (KR) 10-2018-0151783
Nov. 28, 2019 (KR) 10-2019-0155223

(51) Int. Cl. A61K 31/155; A23L 33/10; A61K 31/4409; A61K 31/4375; A61K 31/5377

(52) U.S. Cl. CPC A61K 31/155; A23L 33/10; A61K 31/5377; A61K 31/4375; A61K 31/4409

(57) ABSTRACT

The present invention relates to a pharmaceutical composition for administration in combination with a drug that causes acidosis. Acidosis occurs due to disrupted acid-base balance caused in a case where a large amount of acid is produced and accumulated in the body. There are many cases where acidosis occurs as a side effect associated with drugs for treating various diseases, which poses problems. The pharmaceutical composition of the present invention not only allows the intended purpose, for which a drug that causes acidosis is administered, to be maintained but also has a remarkable effect in decreasing the concentration of acid accumulated in an organism due to administration of the drug that causes acidosis. Thus, the pharmaceutical composition is expected to be widely used in the fields of medicine and health.

### TECHNICAL FIELD

The present invention relates to a pharmaceutical composition for administration in combination with a drug that causes acidosis.

### BACKGROUND ART

Acidosis refers to a condition in which the pH is lower than the normal arterial blood pH of 7.4±0.05 (that is, a condition in which the hydrogen ion concentration is high). Acidosis is classified into “respiratory acidosis” and “metabolic acidosis”, and types of metabolic acidosis include diabetic ketoacidosis, lactic acidosis, or poisoning by toxic substances such as salicylic acid, methanol, and ethylene glycol. Among these, lactic acidosis refers to the acidosis that occurs due to disrupted acid-base balance caused in a case where a large amount of lactic acid is produced and accumulated in the body and is defined as a condition in which lactic acid exceeds 45 mg/dL. In a cell, glucose is metabolized in the presence of oxygen to produce energy. However, in a case where glucose is metabolized in the absence of oxygen, lactic acid is produced. In a case where lactic acidosis continues and acid-base balance is disrupted, symptoms such as muscle weakness, hyperventilation, nausea, vomiting, sweating, or coma may appear and these symptoms may lead to death in severe cases. Therefore, it is important to maintain the acid-base balance by decreasing the concentration of lactic acid that has been over-accumulated in the body. However, there are many cases where lactic acidosis occurs as a side effect associated with drugs for the treatment of various diseases. For example, metformin, which is an antidiabetic drug, is a very effective antidiabetic drug; however, it is known that the most important side effects of metformin are decreased gastrointestinal function and lactic acidosis.

Accordingly, the present invention has been made to solve the above-mentioned problems in the prior art and relates to a pharmaceutical composition for administration in combination with a drug that causes acidosis. The pharmaceutical composition of the present invention not only allows the intended purpose, for which a drug that causes acidosis is administered, to be maintained but also has a remarkable effect in decreasing the concentration of acid accumulated in an organism due to administration of the drug that causes acidosis. Thus, the pharmaceutical composition is expected to be widely used in the fields of medicine and health.

### Technical Problem

The present invention has been made to solve the above-mentioned problems in the prior art and relates to a pharmaceutical composition for administration in combination with a drug that causes acidosis.

However, the technical problem to be achieved by the present invention is not limited to the above-mentioned problems, and other problems that are not mentioned will be clearly understood by those of ordinary skill in the art from the following description.

### Solution to Problem

Hereinafter, various embodiments described herein are described with reference to the drawing. In the following description, numerous specific details are set forth, such as specific configurations, compositions, and processes, etc., in order to provide a thorough understanding of the present invention. However, certain embodiments may be practiced without one or more of these specific details, or in combination with other known methods and configurations. In other instances, well-known processes and manufacturing techniques have not been described in particular detail in order to not unnecessarily obscure the present invention. Reference throughout this specification to “one embodiment” or “an embodiment” means that a particular feature, configuration, composition, or characteristic described in connection with the embodiment is included in at least one embodiment of the invention. Thus, the appearances of the phrase “in one embodiment” or “an embodiment” in various places throughout this specification are not necessarily referring to the same embodiment of the invention. Furthermore, the particular features, configurations, compositions, or characteristics may be combined in any suitable manner in one or more embodiments.

Unless defined otherwise in the specification, all scientific and technical terms as used herein shall have the same meaning as commonly understood by those of ordinary skill in the art to which the present invention belongs.

In an embodiment of the present invention, the term “acidosis” refers to a condition in which the pH is lower than the normal arterial pH of 7.4±0.05 (that is, a condition in which the hydrogen ion concentration is high). Depending on the cause, acidosis is divided into “respiratory acidosis” in which oxygen supply to tissues decreases due to insufficient oxygen absorption in the lungs or decreased blood flow; and “metabolic acidosis” in which the amount of lactic acid increases in the blood or local tissues regardless of the decrease in oxygen level. The causes of respiratory acidosis include shock from bleeding, heart attack, congestive heart failure, pulmonary edema, severe anemia, and the like. Metabolic acidosis is caused by one or more of three mechanisms: acid load, loss of alkali, or impaired renal acid excretion. Examples of a case where acid increases include diabetic ketoacidosis, lactic acidosis, or poisoning by toxic substances such as salicylic acid, methanol, and ethylene glycol.

In an embodiment of the present invention, the term “lactic acidosis” is a type of acidosis and refers to the acidosis that occurs due to disrupted acid-base balance caused in a case where a large amount of lactic acid is produced and accumulated in the body and is defined as a condition in which lactic acid exceeds 45 mg/dL and pH is 7.45 or lower. In a cell, glucose is metabolized in the presence of oxygen to produce energy. However, in a case where glucose is metabolized in the absence of oxygen, lactic acid is produced. In a case where lactic acidosis continues and acid-base balance is disrupted, symptoms such as muscle weakness, hyperventilation, nausea, vomiting, sweating, or coma may appear and these symptoms may lead to death in severe cases. Therefore, it is important to maintain the acid-base balance by decreasing the concentration of lactic acid that has been over-accumulated in the body. The causes of lactic acidosis include liver disease, kidney disease, neurological disease, psychiatric disorder, diabetes, leukemia, acquired immunodeficiency syndrome (AIDS), glycogen storage disease, drugs and poisons, severe infections (systemic sepsis and meningitis), tumors, several genetic metabolic and mitochondrial diseases affecting normal ATP production and muscular dystrophy, and strenuous exercise.

In an embodiment of the present invention, the term “drug that causes lactic acidosis” refers to a drug that causes lactic acidosis in the body as a side effect other than its intended purpose of administration. For example, metformin, which is an antidiabetic drug, causes decreased gastrointestinal function and lactic acidosis as side effects associated with its very effective diabetes therapeutic effect. From the viewpoint that lactic acidosis caused by the drug as mentioned above makes it difficult to freely use the drug for its intended purpose, this causes a huge loss in the health/pharmaceutical industry. Thus, there is a need to develop drugs capable of suppressing a lactic acidosis side effect in the case of being administered in combination with the drug that causes lactic acidosis.

In an embodiment of the present invention, the term “treatment” refers to a set of acts performed for alleviation and/or amelioration of a target disease. For the purpose of the present invention, the treatment includes an act of eliminating the cause of acidosis caused by administration of a drug that causes acidosis; or an act of improving symptoms of acidosis by decreasing the concentration of acid generated, in a case where it is not possible to eliminate the cause.

In an embodiment of the present invention, the term “pharmaceutical composition” refers to a composition administered for a specific purpose. For the purpose of the present invention, the pharmaceutical composition of the present invention is intended to prevent or treat acidosis caused by administration of a drug that causes acidosis and may comprise a compound involved in such prevention or treatment, and a pharmaceutically acceptable carrier, excipient, or diluent. In addition, the pharmaceutical composition according to the present invention comprises an active ingredient of the present invention in an amount of 0.1% to 50% by weight with respect to the total weight of the composition. Examples of carriers, excipients, and diluents, which may be included in the composition of the present invention, may include, but are not limited to, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil.

In an embodiment of the present invention, the term “administration” means introducing the composition of the present invention to a patient by any suitable way, and the composition of the present invention may be administered via any common route as long as the route allows the composition to reach target tissue. Oral administration, intraperitoneal administration, intravenous administration, intramuscular administration, subcutaneous administration, intradermal administration, intranasal administration, intrapulmonary administration, rectal administration, intracavitary administration, intraperitoneal administration, and intrathecal administration may be mentioned; however, the present invention is not limited thereto. In the present invention, an effective amount may be adjusted depending on various factors including the type of disease, the severity of disease, types and contents of the active ingredient(s) and other ingredient(s) included in the composition, type of formulation, the patient's age, weight, general health, sex and diet, frequency of administration, route of administration, secretion rate of the composition, duration of treatment, and simultaneously used drug(s). For adults, the therapeutic pharmaceutical composition may be administered to the body in an amount of 50 ml to 500 ml at a time, in which the dose may be 0.1 ng/kg to 10 mg/kg in a case where the active ingredient is a compound, and may be 0.1 ng/kg to 10 mg/kg in a case where the active ingredient is a monoclonal antibody. The administration may be performed 1 to 12 times a day; and in the case of 12 times a day, the administration may be performed once every 2 hours. In addition, the pharmaceutical composition of the present invention may be administered alone or in combination with other therapies known in the art, such as chemotherapy, radiation, and surgery, for the treatment of target cancer stem cells. In addition, the pharmaceutical composition of the present invention may be administered in admixture with other treatments designed to enhance immune responses, for example, adjuvants or cytokines (or nucleic acids encoding cytokines) as is well known in the art. Other standard delivery methods, such as biolistic transfer or ex vivo treatment, may also be used. In the ex vivo treatment, for example, antigen presenting cells (APCs), dendritic cells, peripheral blood mononuclear cells, or bone marrow cells may be obtained from a patient or an appropriate donor and activated ex vivo with the present pharmaceutical composition, and then returned to the patient.

In an embodiment of the present invention, “food composition” is used in various ways for the prevention or amelioration of indications targeted by the present invention. Food compositions comprising the composition of the present invention as an active ingredient may be prepared in the form of various foods, for example, beverages, gums, tea, vitamin complexes, powders, granules, tablets, capsules, confections, rice cakes, bread, and the like. The food composition of the present invention is composed of ingredients that have been obtained by adding improvements to existing food ingredients having little toxicity and side effects, and thus can be used without worries in a case of being ingested for a long time for preventive purposes. In a case where the composition of the present invention is included in the food composition, the composition of the present invention may be added in an amount corresponding to a proportion of 0.1% to 100% of the total weight. Here, in a case where the food composition is prepared in the form of a beverage, there is no particular limitation except that the beverage contains the food composition at an indicated proportion, and the beverage may contain various flavoring agents or natural carbohydrates, or the like as additional ingredients similarly to conventional beverages. That is, examples of natural carbohydrates may include monosaccharides such as glucose, disaccharides such as fructose, polysaccharides such as sucrose, conventional sugars such as dextrin and cyclodextrin, and sugar alcohol such as xylitol, sorbitol, and erythritol. Examples of the flavoring agents may include natural flavoring agents (thaumatin, stevia extracts (such as rebaudioside A), glycyrrhizin, and the like) and synthetic flavoring agents (saccharin, aspartame, and the like). In addition, the food composition of the present invention may contain various nutrients, vitamins, minerals (electrolytes), flavorings such as synthetic flavorings and natural flavorings, colorants, pectic acid and salts thereof, alginic acid and salts thereof, organic acids, protective colloidal thickeners, pH adjusting agents, stabilizers, preservatives, glycerin, alcohol, carbonizing agents used in carbonated beverages, and the like. These ingredients may be used individually or in combination. The proportion of these additives is usually selected in a range of 0.1 to 100 parts by weight per 100 parts by weight of the composition of the present invention; however, the present invention is not limited thereto.

In an embodiment of the present invention, there is provided a pharmaceutical composition for administration in combination with a drug that causes acidosis, the composition comprising aldehyde dehydrogenase as an active ingredient. In the pharmaceutical composition, the aldehyde dehydrogenase may be at least any one selected from the group consisting of 3-hydroxy-DL-kynurenine, benomyl, cis-diamminedichloridoplatinum (CDDP), chlorpropamide, citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, daidzin, diethylaminobenzaldehyde (DEAB), disulfiram, gossypol, kynurenic acid, molinate, pargyline, phospho(enol)pyruvic acid monosodium salt hydrate, phenylglyoxal, retinoic acid, N-acetyl-N-acetoxy-4-chlorobenzenesulfonamide, and sodium oxamate. In the pharmaceutical composition, the drug that causes acidosis may be administered for treatment of liver disease, kidney disease, neurological disease, psychiatric disorder, diabetes, leukemia, acquired immunodeficiency syndrome (AIDS), glycogen storage disease, infection, tumor, muscular dystrophy, genetic metabolic disorder, mitochondrial disorder, acute dyskinesia, or toxin poisoning. In the pharmaceutical composition, the drug that causes acidosis may be at least any one selected from the group consisting of metformin, phenformin, isoniazid, berberine, and linezolid. In the pharmaceutical composition, the acidosis may be a condition in which the arterial blood pH is 7.35 or lower.

In another embodiment of the present invention, there is provided a food composition for administration in combination with a drug that causes acidosis, comprising aldehyde dehydrogenase as an active ingredient. In the food composition, the aldehyde dehydrogenase may be at least any one selected from the group consisting of 3-hydroxy-DL-kynurenine, benomyl, cis-diamminedichloridoplatinum (CDDP), chlorpropamide, citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, daidzin, diethylaminobenzaldehyde (DEAB), disulfiram, gossypol, kynurenic acid, molinate, pargyline, phospho(enol)pyruvic acid monosodium salt hydrate, phenylglyoxal, retinoic acid, N-acetyl-N-acetoxy-4-chlorobenzenesulfonamide, and sodium oxamate. In the food composition, the drug that causes acidosis may be administered for treatment of liver disease, kidney disease, neurological disease, psychiatric disorder, diabetes, leukemia, acquired immunodeficiency syndrome (AIDS), glycogen storage disease, infection, tumor, muscular dystrophy, genetic metabolic disorder, mitochondrial disorder, acute dyskinesia, or toxin poisoning. In the food composition, the drug that causes acidosis may be at least any one selected from the group consisting of metformin, phenformin, isoniazid, berberine, and linezolid. In the food composition, acidosis may be a condition in which the arterial blood pH is 7.35 or lower.

In yet another embodiment of the present invention, there is provided a method for preventing or treating an acidosis side effect caused by a drug that causes acidosis, comprising administering to a subject in need thereof, a composition that includes aldehyde dehydrogenase as an active ingredient. In the method, the aldehyde dehydrogenase may be at least any one selected from the group consisting of 3-hydroxy-DL-kynurenine, benomyl, cis-diamminedichloridoplatinum (CDDP), chlorpropamide, citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, daidzin, diethylaminobenzaldehyde (DEAB), disulfiram, gossypol, kynurenic acid, molinate, pargyline, phospho(enol)pyruvic acid monosodium salt hydrate, phenylglyoxal, retinoic acid, N-acetyl-N-acetoxy-4-chlorobenzenesulfonamide, and sodium oxamate. In the method, the drug that causes acidosis may be administered for treatment of liver disease, kidney disease, neurological disease, psychiatric disorder, diabetes, leukemia, acquired immunodeficiency syndrome (AIDS), glycogen storage disease, infection, tumor, muscular dystrophy, genetic metabolic disorder, mitochondrial disorder, acute dyskinesia, or toxin poisoning. In the method, the drug that causes acidosis may be at least any one selected from the group consisting of metformin, phenformin, isoniazid, berberine, and linezolid. In the method, the acidosis may be a condition in which the arterial blood pH is 7.35 or lower.

In still yet another embodiment of the present invention, there is provided a composition for use in preventing or treating an acidosis side effect caused by a drug that causes acidosis, the composition comprising aldehyde dehydrogenase as an active ingredient. In the composition, the aldehyde dehydrogenase may be at least any one selected from the group consisting of 3-hydroxy-DL-kynurenine, benomyl, cis-diamminedichloridoplatinum (CDDP), chlorpropamide, citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, daidzin, diethylaminobenzaldehyde (DEAB), disulfiram, gossypol, kynurenic acid, molinate, pargyline, phospho(enol)pyruvic acid monosodium salt hydrate, phenylglyoxal, retinoic acid, N-acetyl-N-acetoxy-4-chlorobenzenesulfonamide, and sodium oxamate. In the composition, the drug that causes acidosis may be administered for treatment of liver disease, kidney disease, neurological disease, psychiatric disorder, diabetes, leukemia, acquired immunodeficiency syndrome (AIDS), glycogen storage disease, infection, tumor, muscular dystrophy, genetic metabolic disorder, mitochondrial disorder, acute dyskinesia, or toxin poisoning. In the composition, the drug that causes acidosis may be at least any one selected from the group consisting of metformin, phenformin, isoniazid, berberine, and linezolid. In the composition, the acidosis may be a condition in which the arterial blood pH is 7.35 or lower.

Hereinafter, the present invention will be described in detail step by step.

### Advantageous Effects of Invention

The present invention relates to a pharmaceutical composition for preventing or treating acidosis. The pharmaceutical composition of the present invention not only allows the intended purpose, for which a drug that causes acidosis is administered, to be maintained but also has a remarkable effect in decreasing the concentration of acid accumulated in an organism due to administration of the drug that causes acidosis. Thus, the pharmaceutical composition is expected to be widely used in the fields of medicine and health.

### BRIEF DESCRIPTION OF DRAWINGS

FIG. 1 illustrates results obtained by identifying, using A549 cells, the effects of administration of candidate substances for acidosis treatment in combination with metformin as a substance that causes acidosis, according to an embodiment of the present invention.

FIG. 2 illustrates results obtained by identifying, using A549 cells, the effects of administration of candidate substances for acidosis treatment in combination with isoniazid as a substance that causes acidosis, according to an embodiment of the present invention.

FIGS. 3A and 3B illustrate results obtained by identifying, using A549 cells, the effects of administration of candidate substances for acidosis treatment in combination with berberine as a substance that causes acidosis, according to an embodiment of the present invention.

FIGS. 4A and 4B illustrate results obtained by identifying, using L132 cells, the effects of administration of candidate substances for acidosis treatment in combination with berberine as a substance that causes acidosis, according to an embodiment of the present invention.

FIGS. 5A and 5B illustrate results obtained by identifying, using A549 cells, the effects of administration of candidate substances for acidosis treatment in combination with linezolid as a substance that causes acidosis, according to an embodiment of the present invention.

FIGS. 6A and 6B illustrate results obtained by identifying, using L132 cells, the effects of administration of candidate substances for acidosis treatment in combination with linezolid as a substance that causes acidosis, according to an embodiment of the present invention.

FIGS. 7A and 7B illustrate results obtained by identifying, using A549 cells, the effects of administration of candidate substances for acidosis treatment in combination with phenformin as a substance that causes acidosis, according to an embodiment of the present invention.

FIGS. 8A and 8B illustrate results obtained by identifying, using L132 cells, the effects of administration of candidate substances for acidosis treatment in combination with phenformin as a substance that causes acidosis, according to an embodiment of the present invention.

### DETAILED DESCRIPTION OF INVENTION

The present inventors screened various candidate substances for aldehyde dehydrogenase inhibitors (ALDH inhibitors) to develop substances for treating acidosis and identified the effects of administration of the candidate substances in combination with drugs for which lactic acidosis is known as a side effect other than their intended pharmaceutical use. As a result, it was found that even for candidate substances for acidosis treatment, which have similar effects of decreasing the lactic acid concentration in a case of being administered alone, such candidate substances exhibit very different effects in a case of being administered in combination with a substance that causes lactic acidosis and the effects vary also depending on the type of substance that causes lactic acidosis.

Hereinafter, the present invention will be described in more detail by way of examples. These examples are only for describing the present invention in more detail, and it will be apparent to those skilled in the art that according to the gist of the present invention, the scope of the present invention is not limited by these examples.

### Example 1: Screening of Candidate Substances for Acidosis Treatment

As a result of screening various candidate substances for aldehyde dehydrogenase inhibitors (ALDH inhibitors) to develop substances for treating acidosis, the present inventors discovered the substances as shown in Table 1 below.

TABLE 1

| No. | Name of substance |
|---|---|
| 1 | 3-Hydroxy-DL-kynurenine (3-HDK) |
| 2 | Benomyl |
| 3 | Cis-diamminedichloridoplatinum (CDDP) |
| 4 | Chlorpropamide |
| 5 | Citral |
| 6 | CVT-10216 (3-[[[3-[4-[(methylsulfonyl)aminolphenyl]-4- |
| | oxo-4H-1-benzopyran-7-yl]oxylmethyl]benzoic acid, or |
| | 3-[[[3-[4-[(methylsulfonyl)amino]phenyl]-4-oxo-4H- |
| | chromen-7-yl]oxy]methyl]benzoic acid) |
| 7 | Cyanamide |
| 8 | Daidzin |
| 9 | Diethylaminobenzaldehyde (DEAB) |
| 10 | Disulfiram |
| 11 | Gossypol |
| 12 | Kynurenic acid |
| 13 | Molinate |
| 14 | Pargyline |
| 15 | Phospho(enol)pyruvic acid monosodium salt hydrate |
| 16 | Phenylglyoxal |
| 17 | Retinoic acid |
| 18 | N-acetyl-N-acetoxy-4-chlorobenzenesulfonamide |
| 19 | Sodium oxamate |

### Example 2: Identification of Effects of Administration of Candidate Substances for Acidosis Treatment in Combination with Substances that Cause Acidosis

As the substance that causes lactic acidosis, the following was used: metformin or phenformin as a biguanide antidiabetic drug; isoniazid as an anti-tuberculosis or anti-depressant; berberine as an antiviral, antifungal, or antibiotic agent; or linezolid as an oxazolidinone antibiotic agent. These drugs are drugs for which lactic acidosis is known as a side effect other than their intended pharmaceutical use.

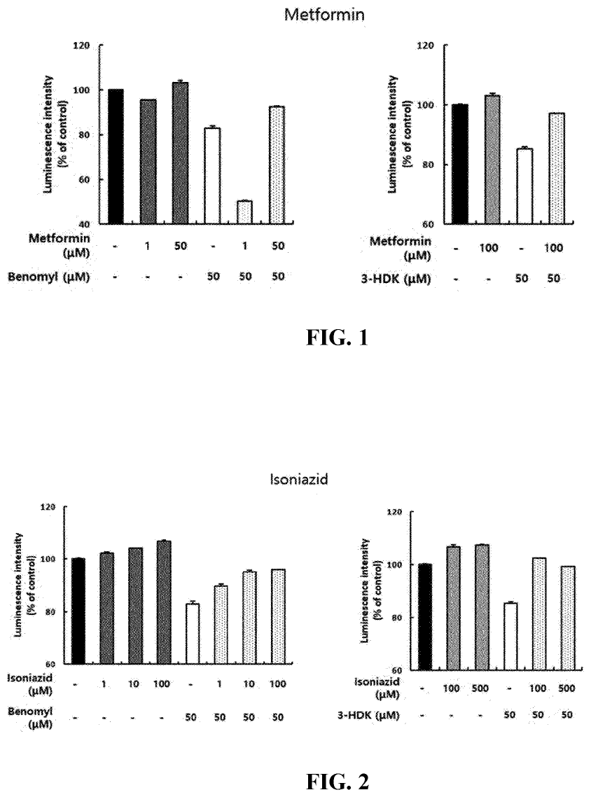

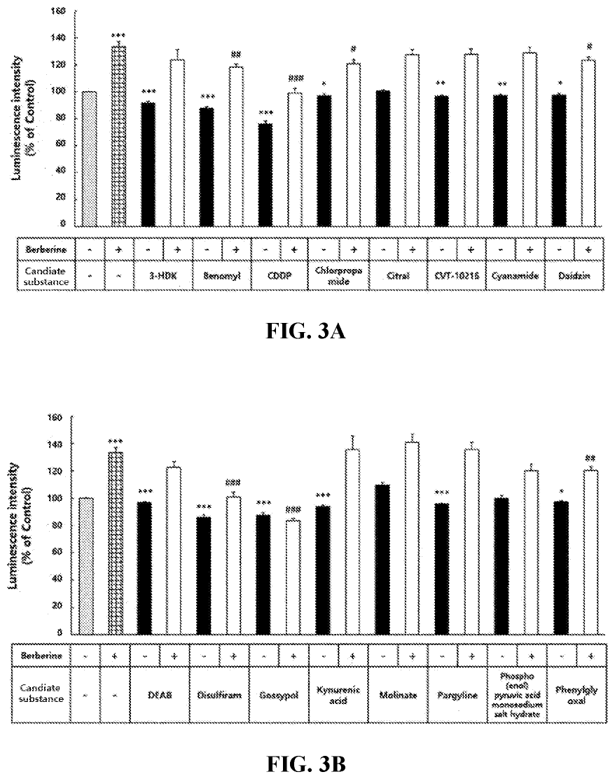

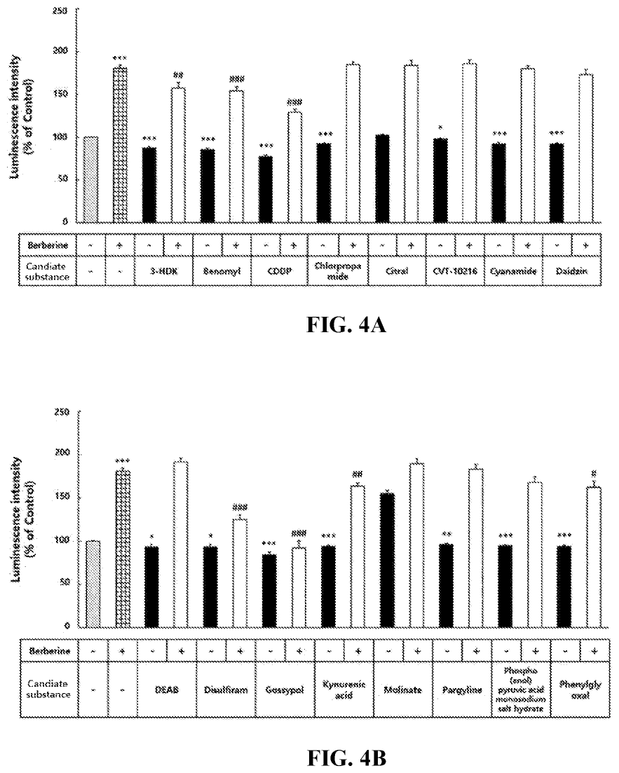

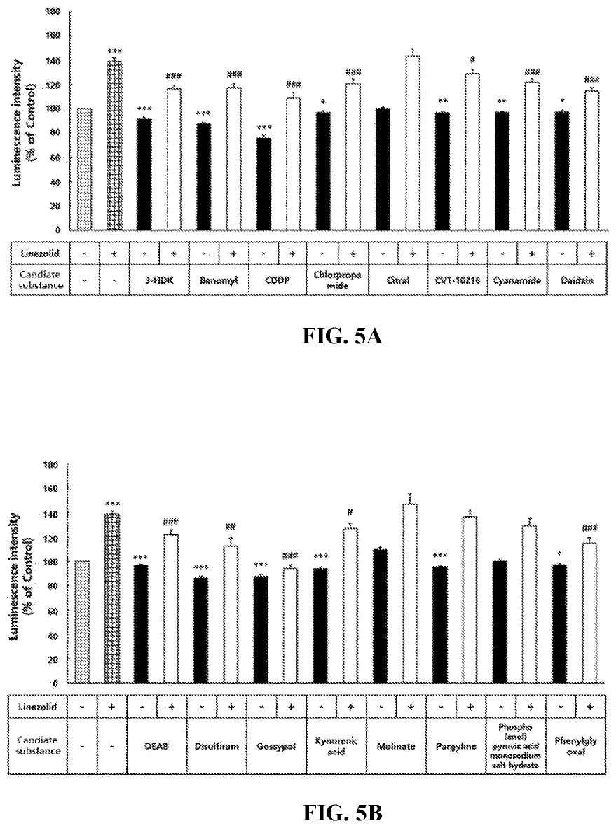

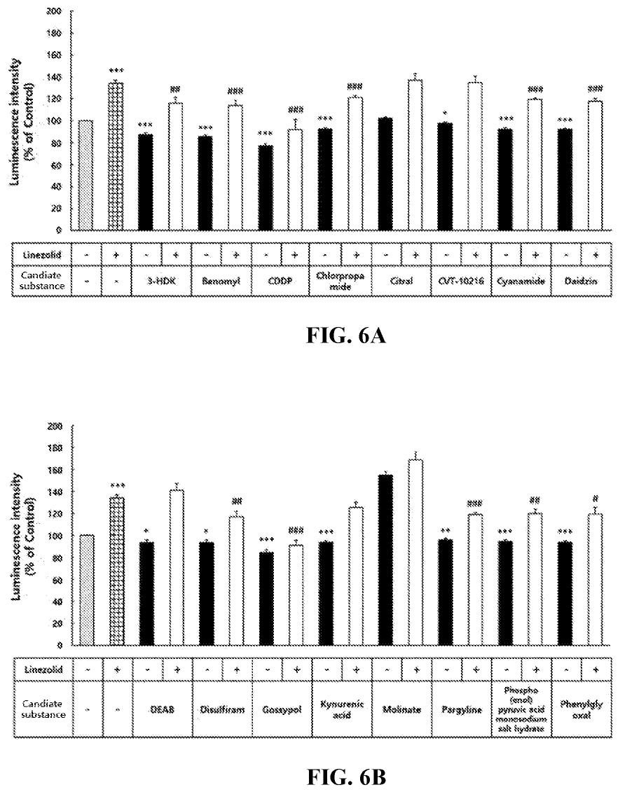

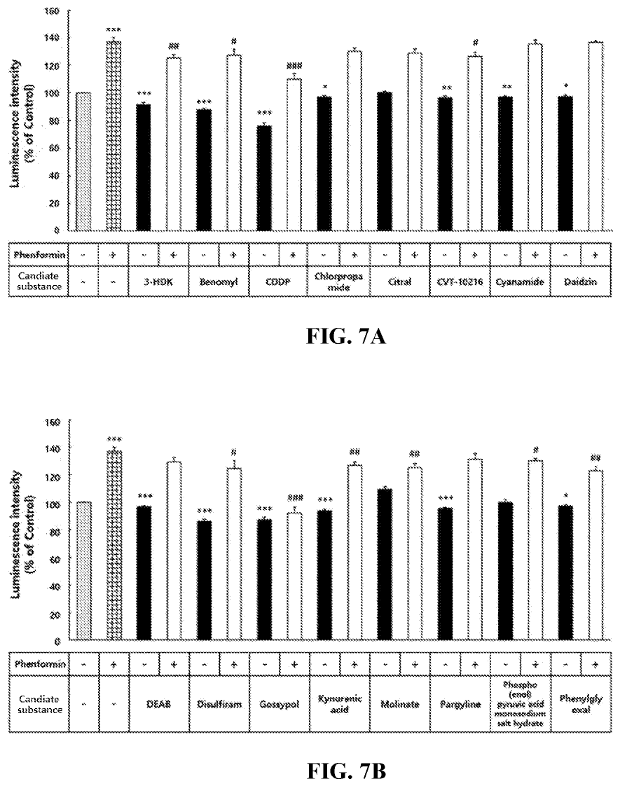

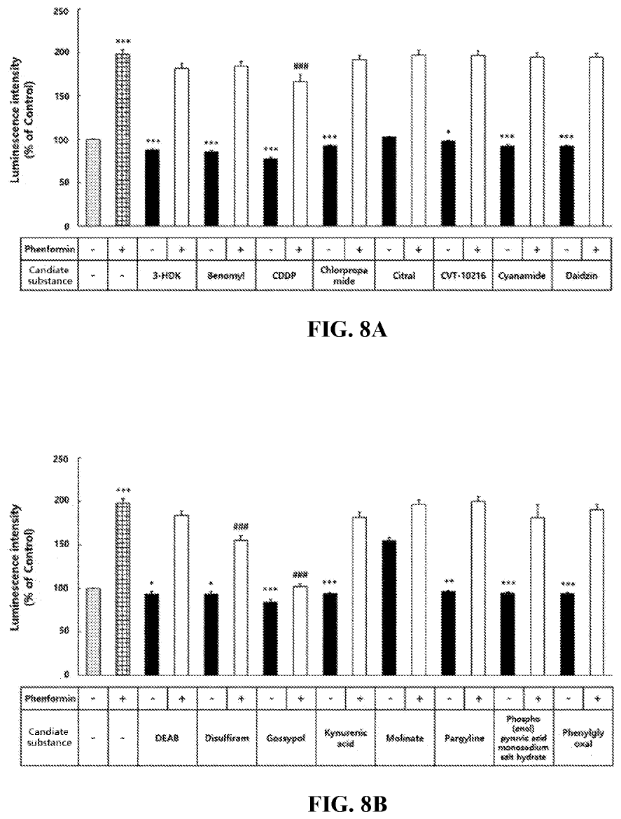

To identify the effects of administration of the candidate substances for lactic acidosis treatment as described in Example 1 in combination with the above-mentioned substances that cause lactic acidosis, A549 cells (cancer cells) or L132 cells (normal cells) were respectively seeded in 96- or 24-well plates at a concentration of 1×10⁴ or 3×10⁵ cells/well, and incubation was performed overnight. Administration of the candidate substance for acidosis treatment in combination with the substance that causes lactic acidosis was performed and incubation was further performed for 24 hours. For the candidate substances for acidosis treatment, each substance was administered at a uniform concentration of 50 μM; and for the substances that cause lactic acidosis, metformin was administered at 1 to 100 μM, isoniazid was administered at 1 to 500 μM, berberine was administered at 10 μM, linezolid was administered at 200 μM, and phenformin was administered at 100 μM. All substances were dissolved in dimethyl sulfoxide (DMSO, Sigma-Aldrich Corporation, St. Louis, Mo., USA), and the substances to be administered in combination were administered simultaneously. Subsequently, 50 μl of the cell culture solution obtained by being diluted with Dulbecco's Phosphate-Buffered Saline (DPBS; Wellgene, Korea), and 50 μl of the lactate assay reaction buffer (Promega, Madison, Wis., USA) were mixed and the mixture was added in a 96-well plate. Reaction was allowed to proceed at room temperature for 1 hour. Then, luminescence was measured with a spectrophotometer (Synergy HTX Multi-Reader; BioTek, Winooski, Vt., USA). At the same time, the number of cells in each sample was measured with a cell viability analysis kit (Cell Counting Kit-8; Dojindo Molecular Technologies, Inc., Kumamoto, Japan), and calculation was performed so that the lactic acid measurement value in each sample could be compared with that in the negative control for the same number of cells. The experimental results are shown in FIGS. 1 to 8, and Tables 2 to 7 below.

TABLE 2

| | | | | Lactic |
|---|---|---|---|---|
| | | | Lactic | acid- |
| | | | acid | de- |
| | Luminescence intensity (%) | | value | creasing |
| | Berberine (10 μM) | | vs. PC | rate vs. |
| Cells: A549 | − | + | (%) | PC (%) |
| Control | 100.00 ± 0.24 | — | — | — |
| (Negative | | | | |
| control, NC) | | | | |
| Substance that | — | 133.35 ± 3.66^{***} | 100.00 | 0.00 |
| causes lactic | | | | |
| acidosis | | | | |
| (Positive | | | | |
| control, PC) | | | | |
| 3-hydroxy-DL- | 91.52 ± 1.46^{***} | 123.42 ± 7.78 | 92.55 | 7.45 |
| kynurenine | | | | |
| Benomyl | 87.71 ± 1.21^{***} | 118.08 ± 2.36^{###} | 88.55 | 11.45 |
| CDDP | 75.98 ± 2.34^{***} | 98.94 ± 3.52^{###} | 74.20 | 25.80 |
| Chlorpropamide | 96.97 ± 1.24^{*} | 120.93 ± 2.88^{#} | 90.69 | 9.31 |
| Citral | 100.48 ± 0.81 | 127.58 ± 3.61 | 95.67 | 4.33 |
| CVT-10216 | 96.46 ± 1.04^{**} | 127.73 ± 4.05 | 95.79 | 4.21 |
| Cyanamide | 97.02 ± 0.81^{**} | 128.76 ± 4.45 | 96.56 | 3.44 |
| Daidzin | 97.42 ± 1.10^{*} | 123.33 ± 2.62^{#} | 92.49 | 7.51 |
| DEAB | 96.72 ± 0.78^{***} | 122.62 ± 4.07 | 91.95 | 8.05 |
| Disulfiram | 86.10 ± 1.66^{***} | 100.91 ± 3.69^{###} | 75.67 | 24.33 |
| Gossypol | 87.59 ± 1.87^{***} | 83.52 ± 1.73^{###} | 62.63 | 37.37 |
| Kynurenic acid | 93.86 ± 1.10^{***} | 135.85 ± 9.82 | 101.87 | −1.87 |
| Molinate | 109.57 ± 1.91 | 141.06 ± 5.84 | 105.78 | −5.78 |
| Pargyline | 95.68 ± 0.70^{***} | 135.87 ± 5.30 | 101.89 | −1.89 |
| Phospho(enol) | 100.07 ± 1.80 | 120.11 ± 4.90 | 90.07 | 9.93 |
| pyruvic acid | | | | |
| monosodium | | | | |
| salt hydrate | | | | |
| Phenylglyoxal | 97.26 ± 1.03^{*} | 120.26 ± 2.93^{##} | 90.18 | 9.82 |

^{*}p < 0.05,
^{**}p < 0.01,
^{***}p < 0.001 (vs. control)
^{#}p < 0.05,
^{##}p < 0.01,
^{###}p < 0.001 (vs. substance that causes lactic acidosis)
Data are presented as the mean ± standard error of the mean (SEM).
Statistical significance was evaluated with Student's t-test.
A p value of < 0.05 was considered statistically significant (n = 10 ≤ 46).

TABLE 3

| | | | | Lactic |
|---|---|---|---|---|
| | | | Lactic | acid- de- |
| | Luminescence intensity (%) | | acid | creasing |
| | Linezolid (200 μM) | | value vs. | rate vs. |
| Cells: A549 | − | + | PC (%) | PC (%) |
| Control | 100.00 ± 0.24 | — | — | — |
| (Negative | | | | |
| control, NC) | | | | |
| Substance that | — | 138.85 ± 2.60^{***} | 100.00 | 0.00 |
| causes lactic | | | | |
| acidosis | | | | |
| (Positive | | | | |
| control, PC) | | | | |
| 3-hydroxy-DL- | 91.52 ± 1.46^{***} | 116.19 ± 2.54^{###} | 83.68 | 16.32 |
| kynurenine | | | | |
| Benomyl | 87.71 ± 1.21^{***} | 117.08 ± 3.87^{###} | 84.32 | 15.68 |
| CDDP | 75.98 ± 2.34^{***} | 108.81 ± 4.46^{###} | 78.37 | 21.63 |
| Chlorpropamide | 96.97 ± 1.24^{*} | 120.54 ± 3.88^{###} | 86.81 | 13.19 |
| Citral | 100.48 ± 0.81 | 143.30 ± 5.38 | 103.20 | −3.20 |
| CVT-10216 | 96.46 ± 1.04^{**} | 129.03 ± 3.60^{#} | 92.93 | 7.07 |
| Cyanamide | 97.02 ± 0.81^{**} | 121.85 ± 2.71^{###} | 87.76 | 12.24 |
| Daidzin | 97.42 ± 1.10^{*} | 114.48 ± 2.81^{###} | 82.45 | 17.55 |
| DEAB | 96.72 ± 0.78^{***} | 121.72 ± 4.16^{###} | 87.66 | 12.34 |
| Disulfiram | 86.10 ± 1.66^{***} | 112.37 ± 6.99^{##} | 80.93 | 19.07 |
| Gossypol | 87.59 ± 1.87^{***} | 94.22 ± 2.91^{###} | 67.86 | 32.14 |
| Kynurenic acid | 93.86 ± 1.10^{***} | 127.10 ± 4.28^{#} | 91.54 | 8.46 |
| Molinate | 109.57 ± 1.91 | 147.02 ± 8.95 | 105.88 | −5.88 |
| Pargyline | 95.68 ± 0.70^{***} | 136.40 ± 5.27 | 98.24 | 1.76 |
| Phospho(enol) | 100.07 ± 1.80 | 129.07 ± 6.22 | 92.96 | 7.04 |
| pyruvic acid | | | | |
| monosodium | | | | |
| salt hydrate | | | | |
| Phenylglyoxal | 97.26 ± 1.03^{*} | 114.89 ± 4.77^{###} | 82.74 | 17.26 |

^{*}p < 0.05,
^{**}p < 0.01,
^{***}p < 0.001 (vs. control)
^{#}p < 0.05,
^{##}p < 0.01,
^{###}p < 0.001 (vs. substance that causes lactic acidosis)
Data are presented as the mean ± standard error of the mean (SEM).
Statistical significance was evaluated with Student's t-test.
A p value of < 0.05 was considered statistically significant (n = 10 ≤ 46).

TABLE 4

| | | | | Lactic |
|---|---|---|---|---|
| | | | Lactic | acid- de- |
| | Luminescence intensity (%) | | acid | creasing |
| | Phenformin (100 μM) | | value vs. | rate vs. |
| Cells: A549 | − | + | PC (%) | PC (%) |
| Control (Negative | 100.00 ± 0.24 | — | — | — |
| control, NC) | | | | |
| Substance | — | 137.16 ± 2.67^{***} | 100.00 | 0.00 |
| that causes | | | | |
| lactic acidosis | | | | |
| (Positive | | | | |
| control, PC) | | | | |
| 3-hydroxy-DL- | 91.52 ± 1.46^{***} | 125.28 ± 2.51^{##} | 91.34 | 8.66 |
| kynurenine | | | | |
| Benomyl | 87.71 ± 1.21^{***} | 127.20 ± 4.47^{#} | 92.74 | 7.26 |
| CDDP | 75.98 ± 2.34^{***} | 109.77 ± 4.37^{###} | 80.03 | 19.97 |
| Chlorpropamide | 96.97 ± 1.24^{*} | 130.12 ± 2.45 | 94.87 | 5.13 |
| Citral | 100.48 ± 0.81 | 128.66 ± 3.38 | 93.80 | 6.20 |
| CVT-10216 | 96.46 ± 1.04^{**} | 126.37 ± 2.97^{#} | 92.13 | 7.87 |
| Cyanamide | 97.02 ± 0.81^{**} | 135.56 ± 2.80 | 98.83 | 1.17 |
| Daidzin | 97.42 ± 1.10^{*} | 136.83 ± 0.93 | 99.76 | 0.24 |
| DEAB | 96.72 ± 0.78^{***} | 129.31 ± 3.07 | 94.28 | 5.72 |
| Disulfiram | 86.10 ± 1.66^{***} | 124.58 ± 5.45^{#} | 90.83 | 9.17 |
| Gossypol | 87.59 ± 1.87^{***} | 92.23 ± 4.44^{###} | 67.24 | 32.76 |
| Kynurenic acid | 93.86 ± 1.10^{***} | 126.98 ± 2.42^{##} | 92.58 | 7.42 |
| Molinate | 109.57 ± 1.91 | 125.00 ± 3.24^{##} | 91.13 | 8.87 |
| Pargyline | 95.68 ± 0.70^{***} | 131.11 ± 4.42 | 95.59 | 4.41 |
| Phospho(enol) | 100.07 ± 1.80 | 130.18 ± 1.81^{#} | 94.91 | 5.09 |
| pyruvic acid | | | | |
| monosodium | | | | |
| salt hydrate | | | | |
| Phenylglyoxal | 97.26 ± 1.03^{*} | 122.91 ± 3.16^{##} | 89.61 | 10.39 |

^{*}p < 0.05,
^{**}p < 001,
^{***}p < 0.001 (vs. control)
^{#}p < 0.05,
^{##}p < 0.01,
^{###}p < 0.001 (vs. substance that causes lactic acidosis)
Data are presented as the mean ± standard error of the mean (SEM).
Statistical significance was evaluated with Student's t-test.
A p value of < 0.05 was considered statistically significant (n = 10 ≤ 46).

TABLE 5

| | | | | Lactic |
|---|---|---|---|---|
| | | | Lactic | acid- de- |
| | Luminescence intensity (%) | | acid | creasing |
| | Berberine (10 μM) | | value vs. | rate vs. |
| Cells: L132 | − | + | PC (%) | PC (%) |
| Control | 100.00 ± 0.29 | — | — | — |
| (Negative | | | | |
| control, NC) | | | | |
| Substance that | — | 180.47 ± 4.06^{***} | 100.00 | 0.00 |
| causes lactic | | | | |
| acidosis | | | | |
| (Positive | | | | |
| control, PC) | | | | |
| 3-hydroxy-DL- | 87.48 ± 1.65^{***} | 157.57 ± 6.63^{##} | 87.31 | 12.69 |
| kynurenine | | | | |
| Benomyl | 85.37 ± 1.55^{***} | 154.09 ± 5.00^{###} | 85.38 | 14.62 |
| CDDP | 76.97 ± 2.13^{***} | 128.90 ± 3.32^{###} | 71.42 | 28.58 |
| Chlorpropamide | 92.44 ± 1.05^{***} | 184.77 ± 3.41 | 102.38 | −2.38 |
| Citral | 102.44 ± 0.98 | 184.14 ± 5.78 | 102.03 | −2.03 |
| CVT-10216 | 97.70 ± 1.10^{*} | 186.15 ± 4.42 | 103.15 | −3.15 |
| Cyanamide | 92.07 ± 1.56^{***} | 180.22 ± 3.21 | 99.86 | 0.14 |
| Daidzin | 91.95 ± 1.14^{***} | 173.14 ± 6.14 | 95.94 | 4.06 |
| DEAB | 93.41 ± 2.71^{*} | 191.48 ± 4.00 | 106.10 | −6.10 |
| Disulfiram | 93.39 ± 2.64^{*} | 124.82 ± 5.30^{###} | 69.16 | 30.84 |
| Gossypol | 84.30 ± 2.90^{***} | 91.96 ± 8.20^{###} | 50.96 | 49.04 |
| Kynurenic acid | 93.70 ± 1.30^{***} | 163.49 ± 3.85^{##} | 90.59 | 9.41 |
| Molinate | 154.71 ± 3.38 | 189.56 ± 5.84 | 105.04 | −5.04 |
| Pargyline | 96.12 ± 1.32^{**} | 183.29 ± 5.41 | 101.56 | −1.56 |
| Phospho(enol) | 94.55 ± 1.37^{***} | 167.93 ± 6.59 | 93.05 | 6.95 |
| pyruvic acid | | | | |
| monosodium | | | | |
| salt hydrate | | | | |
| Phenylglyoxal | 93.72 ± 1.34^{***} | 162.53 ± 7.06^{#} | 90.06 | 9.94 |

^{*}p < 0.05,
^{**}p < 0.01,
^{***}p < 0.001 (vs. control)
^{#}p < 0.05,
^{##}p < 0.01,
^{###}p < 0.001 (vs. substance that causes lactic acidosis)
Data are presented as the mean ± standard error of the mean (SEM).
Statistical significance was evaluated with Student's t-test.
A p value of < 0.05 was considered statistically significant (n = 10 ≤ 46).

TABLE 6

| | | | | Lactic |
|---|---|---|---|---|
| | | | Lactic | acid- |
| | Luminescence intensity (%) | | acid | decreasing |
| | Linezolid (200 μM) | | value vs. | rate vs. |
| Cells: L132 | − | + | PC (%) | PC (%) |
| Control | 100.00 ± 0.29 | — | — | — |
| (Negative | | | | |
| control, NC) | | | | |
| Substance that | — | 134.13 ± 2.76^{***} | 100.00 | 0.00 |
| causes lactic | | | | |
| acidosis | | | | |
| (Positive | | | | |
| control, PC) | | | | |
| 3-hydroxy-DL- | 87.48 ± 1.65^{***} | 115.88 ± 5.40^{##} | 86.39 | 13.61 |
| kynurenine | | | | |
| Benomyl | 85.37 ± 1.55^{***} | 113.78 ± 4.63^{###} | 84.83 | 15.17 |
| CDDP | 76.97 ± 2.13^{***} | 91.59 ± 9.73^{###} | 68.28 | 31.72 |
| Chlorpropamide | 92.44 ± 1.05^{***} | 120.87 ± 2.06^{###} | 90.11 | 9.89 |
| Citral | 102.44 ± 0.98 | 136.84 ± 6.03 | 102.02 | −2.02 |
| CVT-10216 | 97.70 ± 1.10^{*} | 134.51 ± 5.70 | 100.28 | −0.28 |
| Cyanamide | 92.07 ± 1.56^{***} | 119.36 ± 1.28^{###} | 88.99 | 11.01 |
| Daidzin | 91.95 ± 1.14^{***} | 117.76 ± 2.20^{###} | 87.80 | 12.20 |
| DEAB | 93.41 ± 2.71^{*} | 141.12 ± 6.19 | 105.21 | −5.21 |
| Disulfiram | 93.39 ± 2.64^{*} | 116.91 ± 5.09^{##} | 87.16 | 12.84 |
| Gossypol | 84.30 ± 2.90^{***} | 91.00 ± 4.58^{###} | 67.84 | 32.16 |
| Kynurenic acid | 93.70 ± 1.30^{***} | 125.45 ± 4.95 | 93.53 | 6.47 |
| Molinate | 154.71 ± 3.3 | 168.58 ± 7.49 | 125.68 | −25.68 |
| Pargyline | 96.12 ± 1.32^{**} | 118.97 ± 2.06^{###} | 88.70 | 11.30 |
| Phospho(enol) | 94.55 ± 1.37^{***} | 119.78 ± 3.96^{##} | 89.30 | 10.70 |
| pyruvic acid | | | | |
| monosodium | | | | |
| salt hydrate | | | | |
| Phenylglyoxal | 93.72 ± 1.34^{***} | 119.23 ± 6.15^{#} | 88.89 | 11.11 |

^{*}p < 0.05,
^{**}p < 001,
^{***}p < 0.001 (vs. control)
#p < 0.05,
^{##}p < 0.01,
^{###}p < 0.001 (vs. substance that causes lactic acidosis)
Data are presented as the mean ± standard error of the mean (SEM).
Statistical significance was evaluated with Student's t-test.
A p value of < 0.05 was considered statistically significant (n = 10 ≤ 46).

TABLE 7

| | | | | Lactic |
|---|---|---|---|---|
| | | | Lactic | acid- de- |
| | Luminescence intensity (%) | | acid | creasing |
| | Phenformin (100 μM) | | value vs. | rate vs. |
| Cells: L132 | − | + | PC (%) | PC (%) |
| Control | 100.00 ± 0.29 | — | — | — |
| (Negative | | | | |
| control, NC) | | | | |
| Substance that | — | 197.99 ± 4.82^{***} | 100.00 | 0.00 |
| causes lactic | | | | |
| acidosis(Positive | | | | |
| control, PC) | | | | |
| 3-hydroxy-DL- | 87.48 ± 1.65^{***} | 181.68 ± 5.51 | 91.76 | 8.24 |
| kynurenine | | | | |
| Benomyl | 85.37 ± 1.55^{***} | 184.19 ± 5.25 | 93.03 | 6.97 |
| CDDP | 76.97 ± 2.13^{***} | 166.27 ± 8.17^{###} | 83.98 | 16.02 |
| Chlorpropamide | 92.44 ± 1.05^{***} | 191.42 ± 4.96 | 96.68 | 3.32 |
| Citral | 102.44 ± 0.98 | 197.14 ± 5.12 | 99.57 | 0.43 |
| CVT-10216 | 97.70 ± 1.10^{*} | 196.43 ± 5.54 | 99.21 | 0.79 |
| Cyanamide | 92.07 ± 1.56^{***} | 194.40 ± 5.56 | 98.19 | 1.81 |
| Daidzin | 91.95 ± 1.14^{***} | 194.40 ± 4.24 | 98.19 | 1.81 |
| DEAB | 93.41 ± 2.71^{*} | 183.71 ± 5.42 | 92.79 | 7.21 |
| Disulfiram | 93.39 ± 2.64^{*} | 154.84 ± 5.42^{###} | 78.21 | 21.79 |
| Gossypol | 84.30 ± 2.90^{***} | 102.12 ± 2.97^{###} | 51.58 | 48.42 |
| Kynurenic acid | 93.70 ± 1.30^{***} | 181.56 ± 5.82 | 91.70 | 8.30 |
| Molinate | 154.71 ± 3.38 | 196.32 ± 5.38 | 99.16 | 0.84 |
| Pargyline | 96.12 ± 1.32^{**} | 199.74 ± 5.94 | 100.88 | −0.88 |
| Phospho(enol) | 94.55 ± 1.37^{***} | 181.06 ± 15.09 | 91.45 | 8.55 |
| pyruvic acid | | | | |
| monosodium | | | | |
| salt hydrate | | | | |
| Phenylglyoxal | 93.72 ± 1.34^{***} | 190.30 ± 6.09 | 96.12 | 3.88 |

^{*}p < 0.05,
^{**}p < 001,
^{***}p < 0.001 (vs. control)
^{#}p < 0.05,
^{##}p < 0.01,
^{###}p < 0.001 (vs. substance that causes lactic acidosis)
Data are presented as the mean ± standard error of the mean (SEM).
Statistical significance was evaluated with Student's t-test.
A p value of <0.05 was considered statistically significant (n = 10 ≤ 46).

From the above results, it was found that even for candidate substances for acidosis treatment, which have similar effects of decreasing the lactic acid concentration in a case of being administered alone, such candidate substances exhibit very different effects in a case of being administered in combination with a substance that causes lactic acidosis and the effects vary also depending on the type of substance that causes lactic acidosis. For example, in a case where DEAB or disulfiram, which is a candidate substance for acidosis treatment, was administered alone to L132 cells, these substances showed similar lactic acid levels of 93.41±2.71 and 93.39±2.64, respectively, as compared to the negative control group, whereas in a case where DEAB or disulfiram was administered to L132 cells in combination with linezolid, these substances showed remarkably different lactic acid level-decreasing rates which correspond to an increase of 5.21% and a decrease of 12.84%, respectively, as compared to the positive control group.

Although specific parts of the present invention have been described in detail, it is obvious to those skilled in the art that such a specific description is merely a preferred embodiment, and the scope of the present invention is not limited thereto. Therefore, the substantial scope of the present invention will be defined by the appended claims and equivalents thereof.

### INDUSTRIAL APPLICABILITY

Lactic acidosis refers to the acidosis that occurs due to disrupted acid-base balance caused in a case where a large amount of lactic acid is produced and accumulated in the body. In a case where lactic acidosis continues and acid-base balance is disrupted, symptoms such as muscle weakness, hyperventilation, nausea, vomiting, sweating, or coma may appear and these symptoms may lead to death in severe cases. Therefore, it is important to maintain the acid-base balance by decreasing the concentration of lactic acid that has been over-accumulated in the body. However, there are many cases where lactic acidosis occurs as a side effect associated with drugs for the treatment of various diseases. The pharmaceutical composition of the present invention not only allows the intended purpose, for which a drug that causes acidosis is administered, to be maintained but also has a remarkable effect in decreasing the concentration of acid accumulated in an organism due to administration of the drug that causes acidosis. Thus, the pharmaceutical composition is expected to be widely used in the fields of medicine and health.

1-20. (canceled)

21. A method for preventing or treating an acidosis side effect in a subject caused by a drug while being administered to the subject for treating a disease as intended, the method comprising administering to the subject a pharmaceutical composition in combination with the drug, wherein the pharmaceutical composition comprises an aldehyde dehydrogenase inhibitor.

22. The method of claim 21, wherein the aldehyde dehydrogenase inhibitor is selected from the group consisting of 3-hydroxy-DL-kynurenine, benomyl, cis-diamminedichloridoplatinum (CDDP), chlorpropamide, citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, daidzin, diethylaminobenzaldehyde (DEAB), disulfiram, gossypol, kynurenic acid, molinate, pargyline, phospho(enol)pyruvic acid monosodium salt hydrate, phenylglyoxal, retinoic acid, N-acetyl-N-acetoxy-4-chlorobenzenesulfonamide, and sodium oxamate.

23. The method of claim 21, wherein the drug is administered for treating a liver disease, kidney disease, neurological disease, psychiatric disorder, diabetes, leukemia, acquired immunodeficiency syndrome (AIDS), glycogen storage disease, infection, tumor, muscular dystrophy, genetic metabolic disorder, mitochondrial disorder, acute dyskinesia, or toxin poisoning.

24. The method of claim 23, wherein the drug is metformin, phenformin, isoniazid, berberine, or linezolid.

25. The method of claim 23, wherein the toxin is salicylic acid, methanol, or ethylene glycol.

26. The method of claim 21, wherein the acidosis side effect is indicated by arterial blood pH of 7.35 or lower.

27. The method of claim 21, wherein the pharmaceutical composition further comprises a pharmaceutically acceptable carrier.

28. The method of claim 27, wherein the pharmaceutically acceptable carrier is lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, or mineral oil.

29. The method of claim 21, wherein the aldehyde dehydrogenase inhibitor is present in the pharmaceutical composition in an amount of about 0.1% to about 50% by weight.

30. The method of claim 21, wherein the aldehyde dehydrogenase inhibitor is administered to the subject at a dose of about 0.1 ng/kg to about 10 mg/kg of body weight.

31. The method of claim 21, wherein the pharmaceutical composition is administered 1 to 12 times a day.

32. The method of claim 21, wherein the pharmaceutical composition is administered orally, intraperitoneally, intravenously, intramuscularly, subcutaneously, intradermally, intranasally, rectally, intraperitoneally, intrathecally, via intrapulmonary administration, or via intracavitary administration.

33. A method for treating diabetes in a subject in need thereof, the method comprising administering to the subject a therapeutically effective amount of an antidiabetic drug in combination with an aldehyde dehydrogenase inhibitor.

34. The method of claim 33, wherein the antidiabetic drug is metformin or phenformin.

35. The method of claim 33, wherein the aldehyde dehydrogenase inhibitor is selected from the group consisting of 3-hydroxy-DL-kynurenine, benomyl, cis-diamminedichloridoplatinum (CDDP), chlorpropamide, citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, daidzin, diethylaminobenzaldehyde (DEAB), disulfiram, gossypol, kynurenic acid, molinate, pargyline, phospho(enol)pyruvic acid monosodium salt hydrate, phenylglyoxal, retinoic acid, N-acetyl-N-acetoxy-4-chlorobenzenesulfonamide, and sodium oxamate.

36. A method for treating an infection in a subject in need thereof, the method comprising administering to the subject a therapeutically effective amount of an antiviral, antifungal, or antibiotic drug in combination with an aldehyde dehydrogenase inhibitor.

37. The method of claim 36, wherein the antiviral, antifungal, or antibiotic drug is isoniazid, berberine, or linezolid.

38. The method of claim 36, wherein the infection is tuberculosis and the antibiotic drug is isoniazid.

39. The method of claim 36, wherein the aldehyde dehydrogenase inhibitor is selected from the group consisting of 3-hydroxy-DL-kynurenine, benomyl, cis-diamminedichloridoplatinum (CDDP), chlorpropamide, citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, daidzin, diethylaminobenzaldehyde (DEAB), disulfiram, gossypol, kynurenic acid, molinate, pargyline, phospho(enol)pyruvic acid monosodium salt hydrate, phenylglyoxal, retinoic acid, N-acetyl-N-acetoxy-4-chlorobenzenesulfonamide, and sodium oxamate.
